(19) United States
(12) Patent Application Publication
(10) Pub. No.: US 2012/0307060 A1
(43) Pub. Date: Dec. 6, 2012

(54) Bicycle Racing Apparatus

(76) Inventor: Neal Henderson, Escondido, CA (US)

(21) Appl. No.: 13/488,160

(22) Filed: Jun. 4, 2012

Related U.S. Application Data
(60) Provisional application No. 61/492,505, filed on Jun. 2, 2011.
(60) Provisional application No. 61/570,093, filed on Dec. 13, 2011.

(51) Int. Cl. H04N 7/18; A41D 13/00; A61F 9/02; A43B 5/14

(52) U.S. Cl. 348/148; 36/131; 2/69; 2/431; 348/E07.085

(57) ABSTRACT

The present invention comprises a suite of innovations that reduce the coefficient of drag of a bicycle rider. The innovations include aerodynamically shaped bicycle riding shoes, an inflatable body fairing worn on the bicyclist's chest area that directly reduces the coefficient of drag of the bicyclist's torso while in an aerodynamically efficient riding position, and a riding goggle and a portable imaging system that reduce the coefficient of drag of a bicycle rider by allowing the athlete to assume and maintain a more aerodynamically efficient riding position.

### CROSS-REFERENCE TO RELATED APPLICATIONS

This application claims the benefit of U.S. Provisional Patent Applications 61/492505, filed Jun. 2, 2011, entitled “Bicycle Racing Apparatus” and U.S. Provisional Patent Applications 61/570093, filed Dec. 13, 2011, also entitled “Bicycle Racing Apparatus”, currently pending, by the same inventor and hereby incorporated by reference.

### BACKGROUND OF THE INVENTION

1. Technical Field

The present invention relates to articles of apparel and related devices that enable the rider of a bicycle to assume and maintain a more nearly ideal aerodynamic configuration as would be particularly beneficial to a rider participating in a bicycle racing event.

2. Related Background Art

The technologies underpinning bicycle racing have evolved rapidly over the past few decades resulting in new materials and devices for increasing the speed and efficiency of the racing system comprising the machine and its rider. The primary aims of these development activities have been two-fold: 1) reducing the weight of a system element while maintaining minimum structural strength requirements and 2) reducing the aerodynamic drag of the racing system. In spite of these advances, the rider remains the single largest source of inefficiency, typically representing more than 60% of the total aerodynamic drag on the racing system. The bicycle itself typically represents only about 20% of the total drag, with the balance attributed to other mechanisms.

The physics of bicycle movement require that the aerodynamic drag force be proportional to the density of the surrounding fluid (air), the square of the velocity through the fluid, and the sum of the drag coefficients related to the rider and the bicycle. The drag coefficients are, in turn, related to the product of a dimensionless coefficient of drag that relates to the shape of an object and that object's frontal area exposed to the moving fluid stream. Thus, to reduce the aerodynamic drag force at a particular vehicle speed, one is led to reduce either or both of the frontal area and the coefficient of drag of the rider and the bicycle. Such has been the motivation behind the development of devices such as aerodynamically shaped frames, handle bars, wheels, brakes, etc. that act primarily to reduce the coefficient of drag of those elements of the bicycle. Similarly, the development of aerodynamically shaped helmets, skin suits, glasses and the like reduce the coefficient of drag of the rider.

Much improvement has been accomplished using these devices, but the nature of athletic competition is that all highly skilled bicyclists train to benchmarks set by recent competitions, and arrive at a new race with remarkably similar physical capabilities. Improvements of a few percentage points attributable to their equipment can easily make the difference between winning and losing an important competition. Consequently, there is an ongoing need for innovation in this area, and a focus on reducing the coefficient of drag of the rider would seem to be the most profitable direction.

The present invention comprises a suite of innovations that reduce the coefficient of drag of the bicycle rider by either directly improving his aerodynamic profile or by allowing the athlete to assume and maintain a more aerodynamically efficient riding position.

DISCLOSURE OF THE INVENTION

The elements of the present invention include two items of apparel that directly reduce the coefficient of drag of a bicycle rider and two devices that allow the rider to maintain a riding position that further reduces the coefficient of drag of the rider. The first of the items of apparel is a pair of bicycle racing shoes that includes an aerodynamic molding over the usual pedal coupling that is typically attached directly to the sole of the shoe. The molding directly reduces the coefficient of drag of the shoe and has the additional benefit of making it easier for the bicyclist to walk in the shoes without slipping and inflicting possible injury to the bicyclist. The second item of apparel is an inflatable body fairing worn on the bicyclist's chest area that directly reduces the coefficient of drag of the bicyclist's torso while in an aerodynamically efficient riding position. The fairing has the additional benefit of being capable of being inflated with water and food/electrolytes in separate compartments that can be used to nourish the bicyclist during the race with different fluids as desired.

The third device is a unique configuration for a riding goggle that incorporates a means of providing forward vision to allow the bicyclist to follow the race course and avoid obstacles while the bicyclist's head is lowered to assume a more aerodynamically efficient riding position and help prevent fatigue of the neck muscles. The fourth device is a unique vision system for bicycle racers that comprises a miniature, low power video camera attached to the rear surface of a racing helmet so as to provide a field of vision extending ahead of the racer when the racer's head is lowered into an aerodynamically efficient position. In a preferred embodiment, the image signal from the video camera is directed to a suitable display unit either wirelessly or by using suitable lightweight electronic cables. In one embodiment the display unit is a miniature video display such as typically used as a viewfinder in a modern electronic camera system that is directly attached to a specially modified racing eyewear so as to be within the bicyclist's direct field of vision. In another embodiment the display unit is a larger video display mechanically attached to the front frame of the bicycle so that it is within the field of view of the bicyclist when the bicyclist's head is lowered to assume a more aerodynamically efficient riding position.

### BRIEF DESCRIPTION OF THE DRAWINGS

FIG. 1 shows a bicycle rider in a conventional riding position.

FIG. 2 shows a bicycle rider in an improved riding position and also employing elements of the present invention.

FIG. 3 shows a side view of a conventional bicycle racing shoe.

FIG. 4 shows a bottom view of a conventional bicycle racing shoe.

FIG. 5 shows a side view of an improved bicycle racing shoe.

FIG. 6 shows a bottom view of an improved bicycle racing shoe.

FIG. 7 shows a frontal view of an embodiment of an inflatable body fairing.

FIG. 8 shows a side view of an embodiment of an inflatable body fairing.

FIG. 9 shows a side view of a different embodiment of an inflatable body fairing.

FIG. 10 shows a frontal view of an embodiment of an improved racing goggle.

FIG. 11 shows a side view of an embodiment of an improved racing goggle.

FIG. 12 shows a side view of an embodiment of an improved racing goggle as worn.

FIG. 13 shows a frontal view of a different embodiment of an improved racing goggle.

FIG. 14 shows a side view of a different embodiment of an improved racing goggle.

FIG. 15 shows a side view of a different embodiment of an improved racing goggle as worn.

FIG. 16 shows a side view of a first embodiment of the vision system wherein video camera and video display elements of the invention are both mounted on a bicyclist's helmet.

FIG. 17 shows a front view of the helmet-mounted video display element of the invention shown in FIG. 16.

FIG. 18 shows a side view of a second embodiment of the vision system including helmet-mounted video camera and eyewear-mounted video display elements of the invention.

FIG. 19 shows a front and right side view of the embodiment of the eyewear-mounted video display element of the invention shown in FIG. 18.

FIG. 20 shows a side view of a third embodiment of the invention in which the video display is mounted on the bicycle frame.

### DETAILED DESCRIPTION

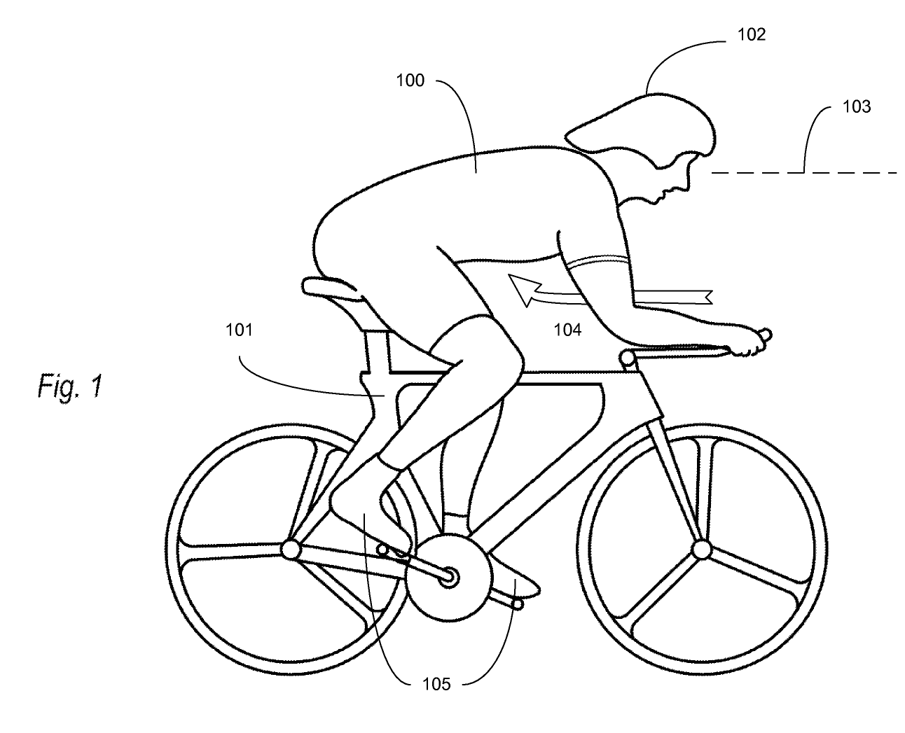

FIG. 1 depicts the prior art and shows a rider 100 on a bicycle 101 in a conventional racing configuration in which the rider's head 102 must be raised most of the time to provide forward vision along line of sight 103 to follow the race course and to avoid collision with obstacles. When the head is raised, the chest is typically also raised, thereby increasing the effective frontal area 104 of the torso and the coefficient of drag of the rider. Conventional bicycle racing shoes 105 have flat soles that generate turbulence that contributes to the coefficient of drag of the rider.

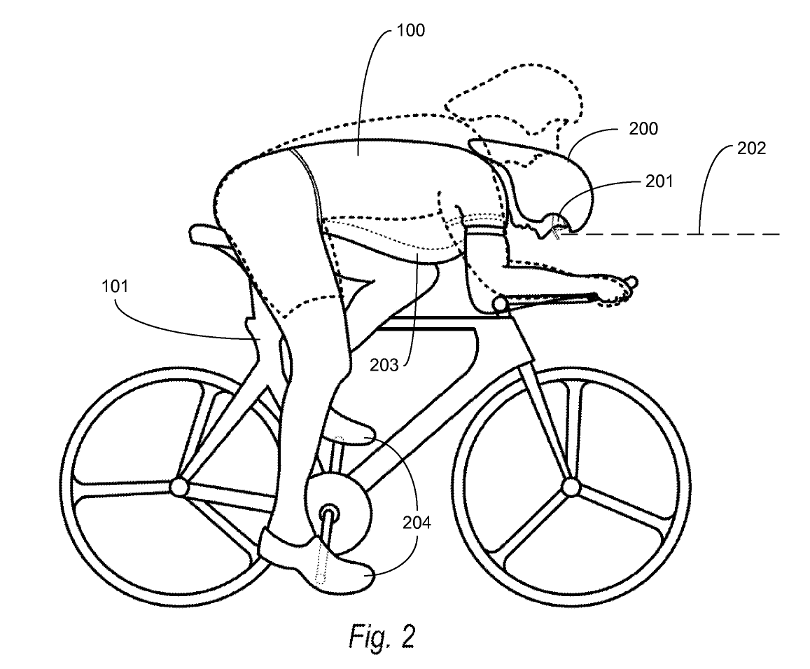

FIG. 2 shows a rider 100 on a bicycle 101 in a more aerodynamically efficient riding position wherein the rider's head 200 and torso are lowered to reduce the effective frontal area and achieve a configuration having a smaller coefficient of drag. The conventional racing configuration is shown in dotted lines for reference. The bicyclist is wearing special eyewear 201 that provides a forward line of sight 202 while in this riding position in order for the bicyclist to follow the race course and avoid obstacles. Additionally, the bicyclist is wearing an inflatable body fairing 203 that reduces the coefficient of drag contributed by the bicyclist's torso, and racing shoes having aerodynamically shaped soles that reduce the coefficient of drag contributed by the shoes.

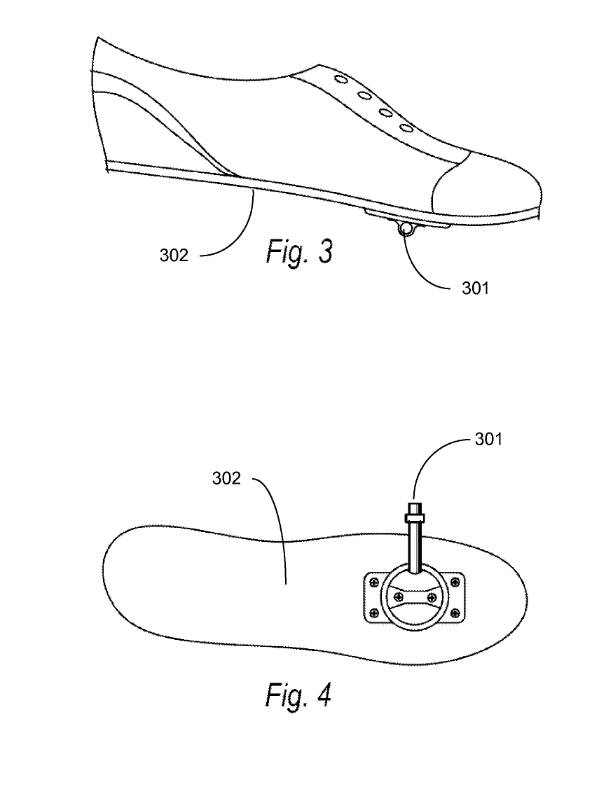

FIGS. 3 and 4 illustrate the prior art in bicycle racing shoes in which pedal coupling members 301 are attached directly to the flat soles 302 of each shoe. The flat soles generate turbulence that increases the coefficient of drag contributed by the shoes. Additionally, the metal pedal coupling members 301 can make footing treacherous while attempting to walk in these shoes, resulting in slipping and potential injury from a fall.

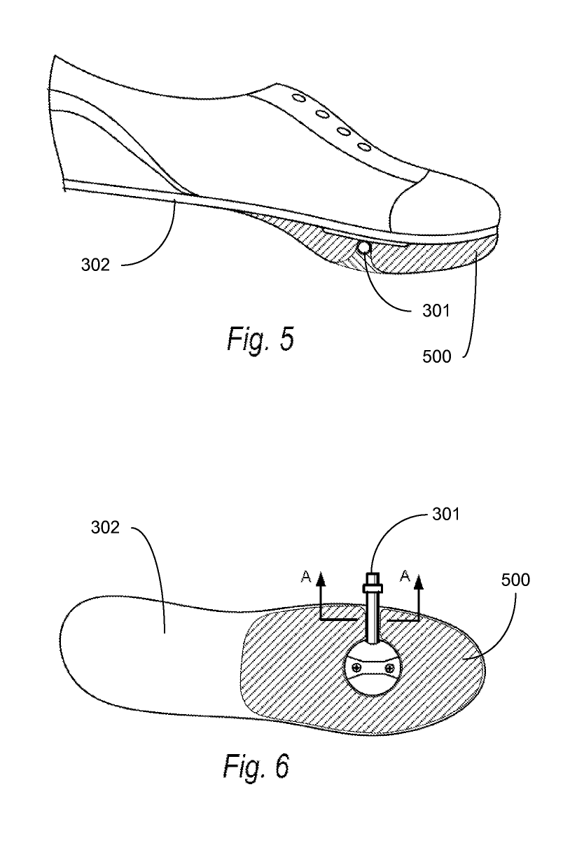

FIGS. 5 and 6 show the improved racing shoe in which a molding compound is used to form an aerodynamically shaped surface 500 on at least the front half of the sole 302 around the pedal coupling member 301. Nonlimiting examples of the molding compound are materials such as an epoxies, crepe sole shoe materials, urethane compounds and room temperature vulcanizing silicone compounds. This aerodynamically shaped surface reduces the coefficient of drag contributed by the shoes and further makes it easier to walk in the shoes without risking injury due to slipping since the molding compound completely surrounds the metal pedal coupling member 301.

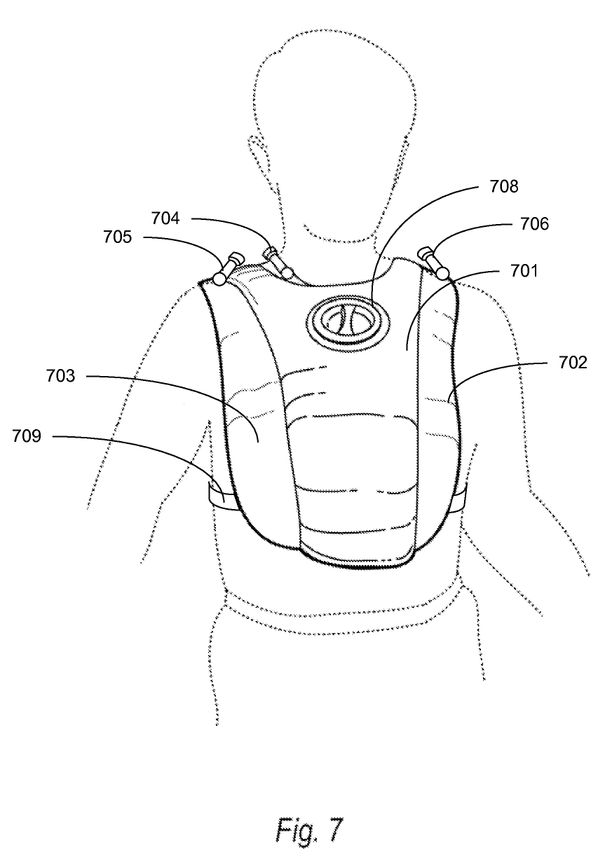

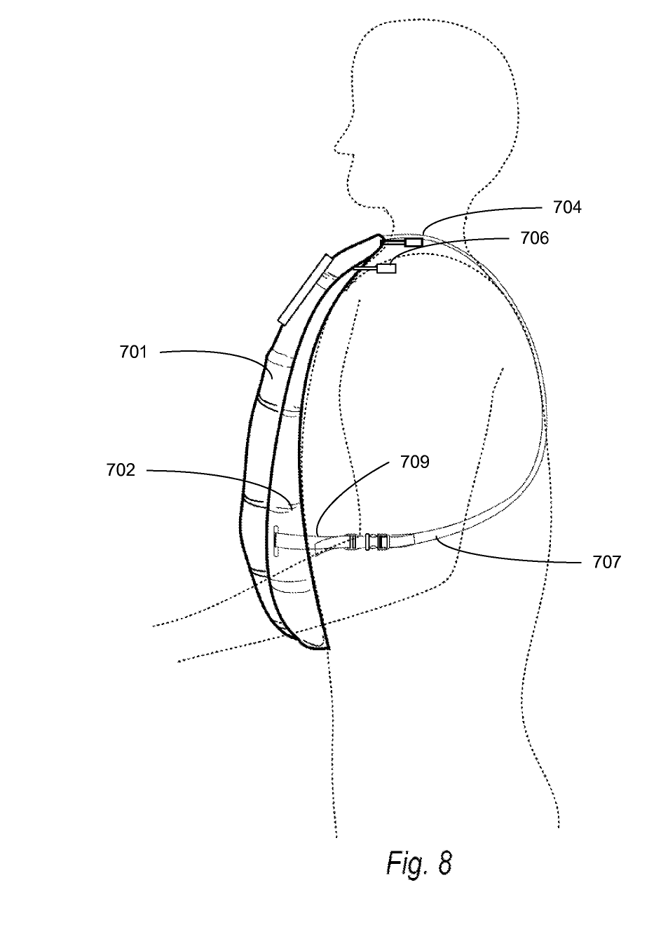

FIG. 7 shows a frontal view of an inflatable body fairing used to provide an enhanced aerodynamic shape that reduces the coefficient of drag contributed by the bicyclist's torso. The body fairing includes a central inflatable section 701 and separate side inflatable sections on the left 702 and right 703 sides of the bicyclist's frontal torso, each of which is inflated through a separate integrated coupling and valve assembly 704, 705, and 706. Although nominally inflated with air, the sections 701-703 can alternatively be inflated with water and food/electrolytes that can be used to nourish the bicyclist during the race. In the figure, the central section 701 is equipped with an anti-splash opening having a quick fill cap 708 that allows for rapid filling of the section with water or electrolyte/food material during a race. FIG. 8 shows a left side view of one embodiment of the body fairing, illustrating the reduced thickness of the side section 702 compared with central section 701. The body fairing is attached to the torso using shoulder straps 707 connected at the top of the body fairing that attach to waist strap 709 connected at the bottom of the fairing and is intended to be worn beneath a conventional racing shirt.

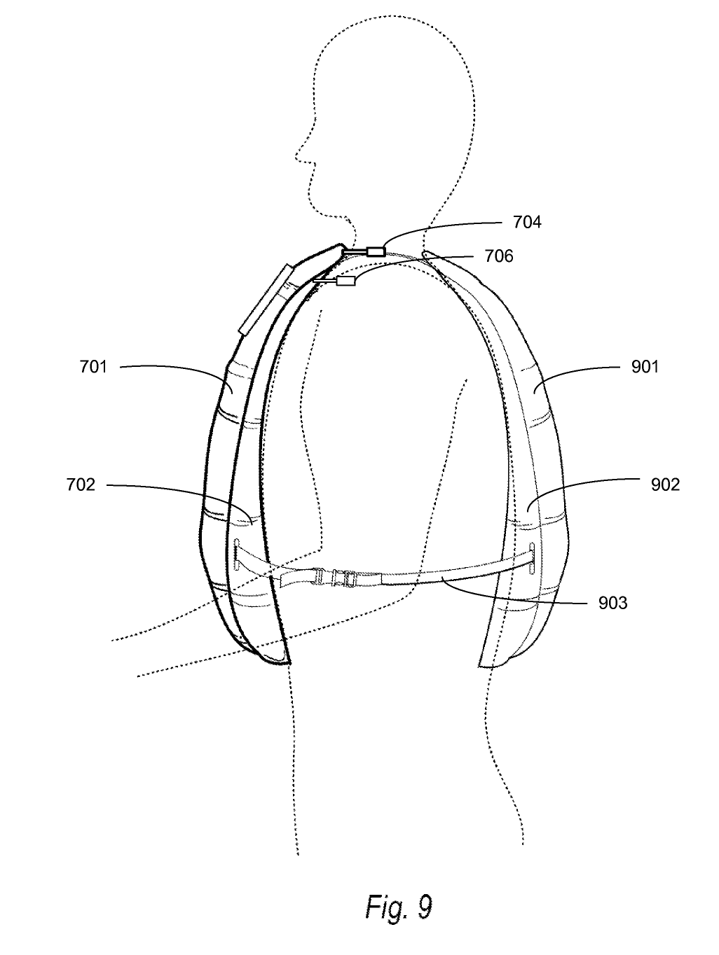

FIG. 9 shows a left side view of an alternate embodiment that includes rear central section 901 and rear side sections, of which rear left side section 902 is shown. The body fairing is attached to the torso using waist straps 903 and is intended to be worn beneath a conventional racing shirt.

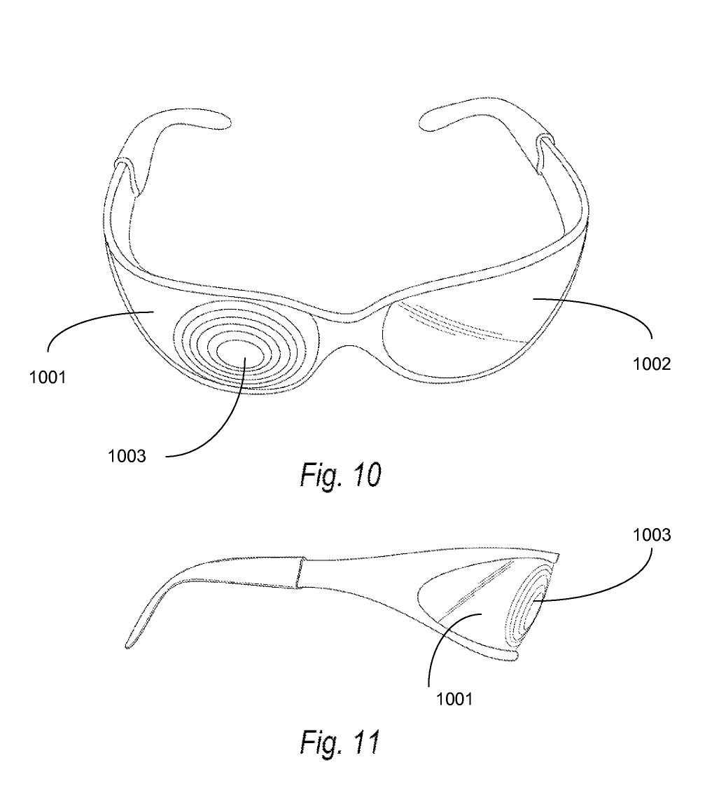

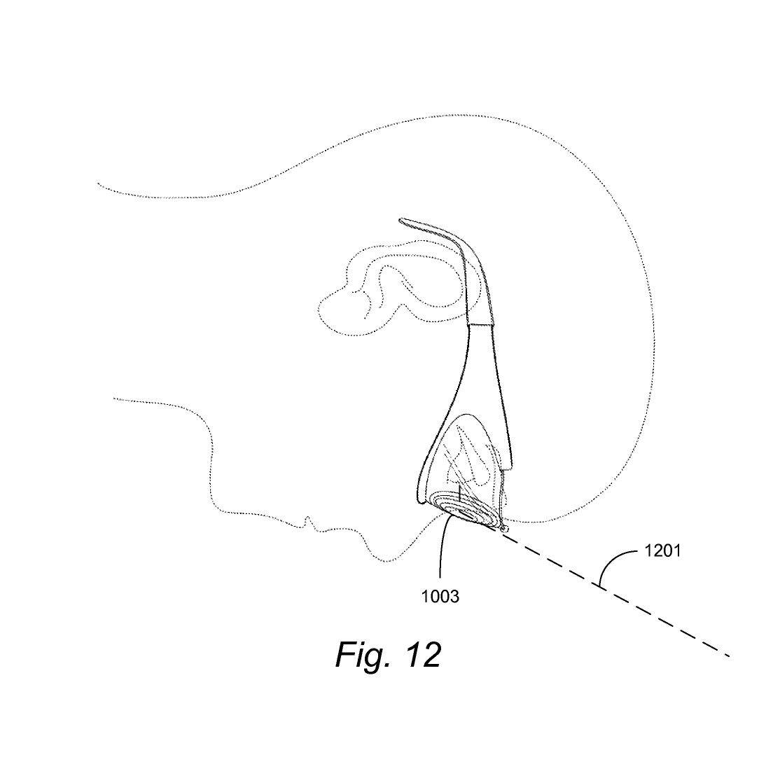

FIGS. 10 and 11 show frontal and right side views, respectively, of a special bicycle racing goggle designed to provide a rectified forward vision while the bicyclist's head faces downward in the more aerodynamically efficient riding position shown in FIG. 2. The goggle includes right 1001 and left 1002 transparent lenses, and an optical device 1003 in the form of a wide-angle lens implemented as a Fresnel lens and integrated into at least one of the lenses 1001 and 1002. FIG. 12 shows a right side view the racing goggle of FIGS. 10 and 11 in an as-worn configuration and illustrates the forward line of sight 1201 provided by the Fresnel lens 1003.

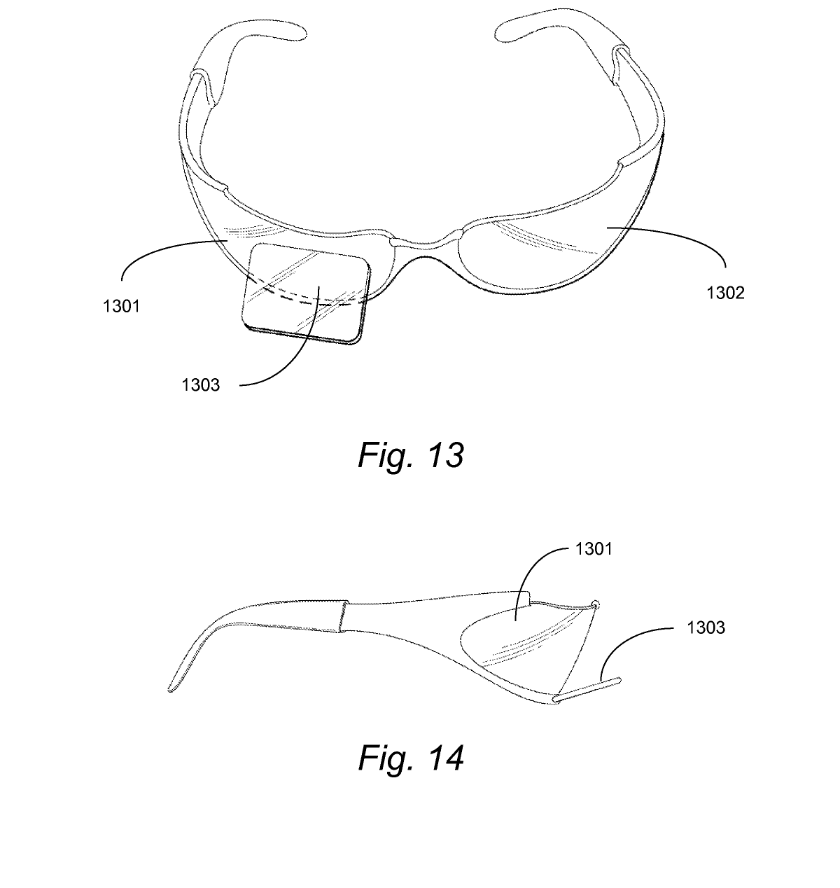

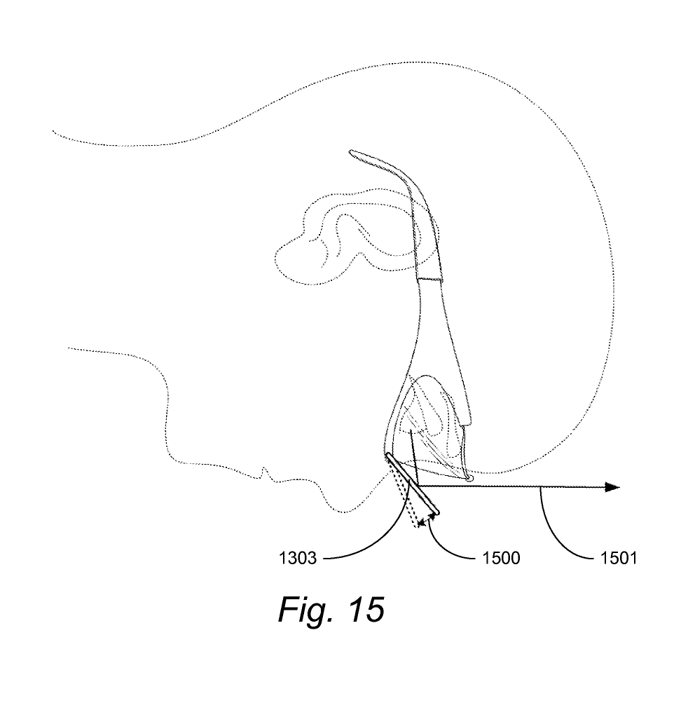

FIGS. 13 and 14 show frontal and right side views, respectively, of an alternate embodiment of a special bicycle racing goggle designed to provide a measure of forward vision while the bicyclist's head faces downward in the more aerodynamically efficient riding position shown in FIG. 2. The goggle includes right 1301 and left 1302 transparent lenses, and an optical device 1303 in the form of a prism integrated into at least one of the lenses. FIG. 15 shows a right side view the racing goggle of FIGS. 13 and 14 in an as-worn configuration and illustrates the forward line of sight 1501 provided by the prism 1303 which angle can be adjusted through an adjustment range 1500.

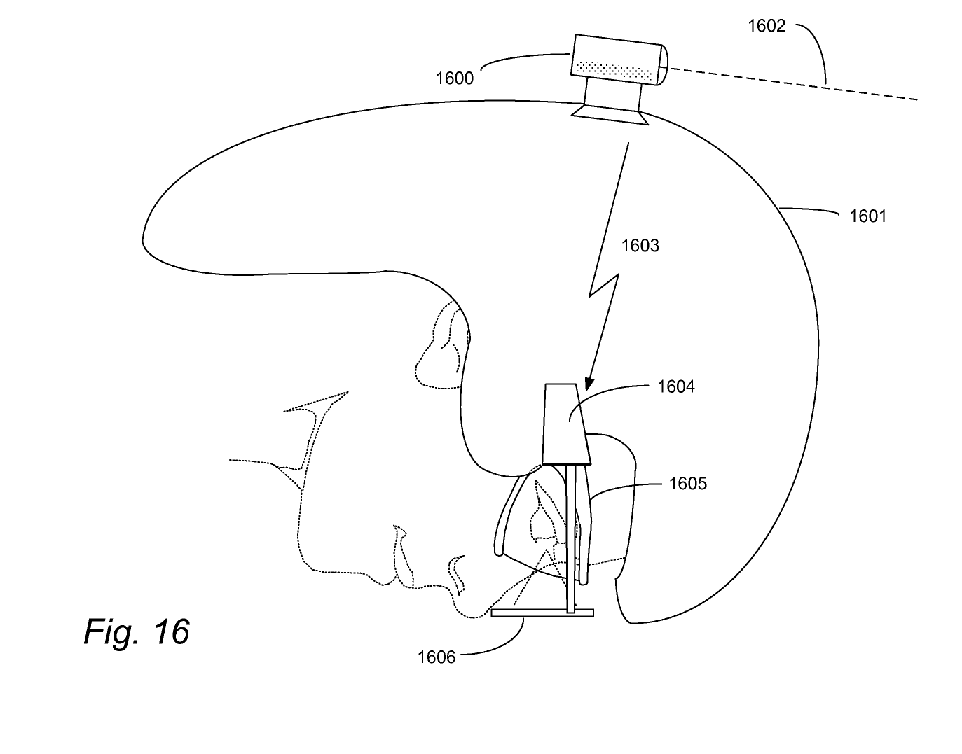

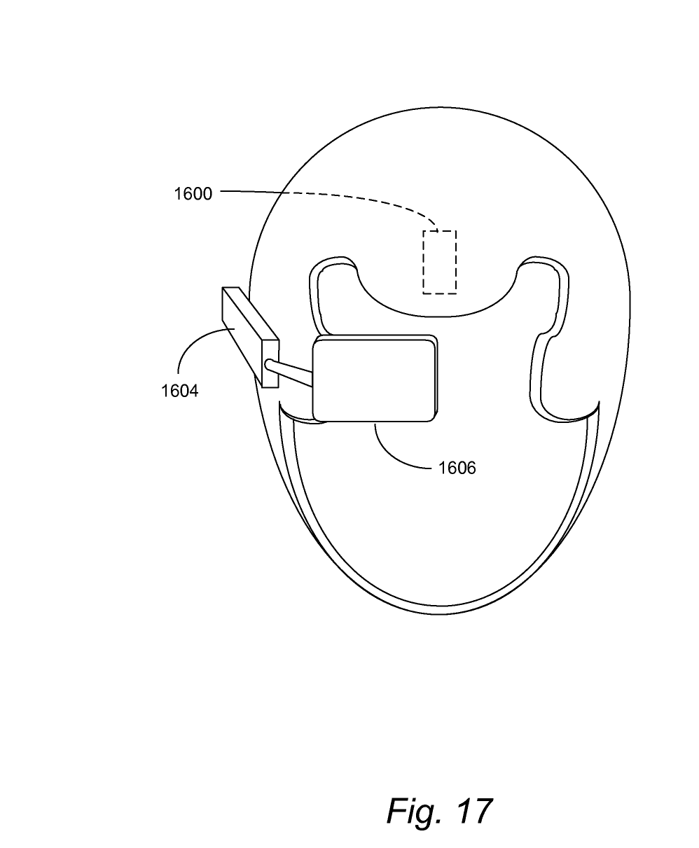

FIG. 16 shows an embodiment comprising a miniature battery-operated video camera 1600 mounted to a racing helmet 1601 to provide a forward field of view 1602 when the racer's head is lowered into an aerodynamically efficient position. The video camera is constructed using any of the available low power imager chips that are used in portable cellular “smart” telephone units, for example. In a non-limiting example, the image signal from the camera 1603 is wirelessly communicated using additional communications hardware and software enclosed in the camera housing to a battery-operated receiver/processor 1604 also mounted on the helmet. The received image is displayed on a miniature video display device 1606 that presents the forward scene directly to the bicyclist's eye. The miniature video display device is a low-power unit such as is typically used as a viewfinder in a modern electronic camera system. The bicyclist wears conventional racing eyewear 1605.

FIG. 17 shows a front view of the helmet-mounted video display embodiment shown in FIG. 16 in order to more clearly illustrate the placement of the receiver/processor 1604 and video display 1606 elements.

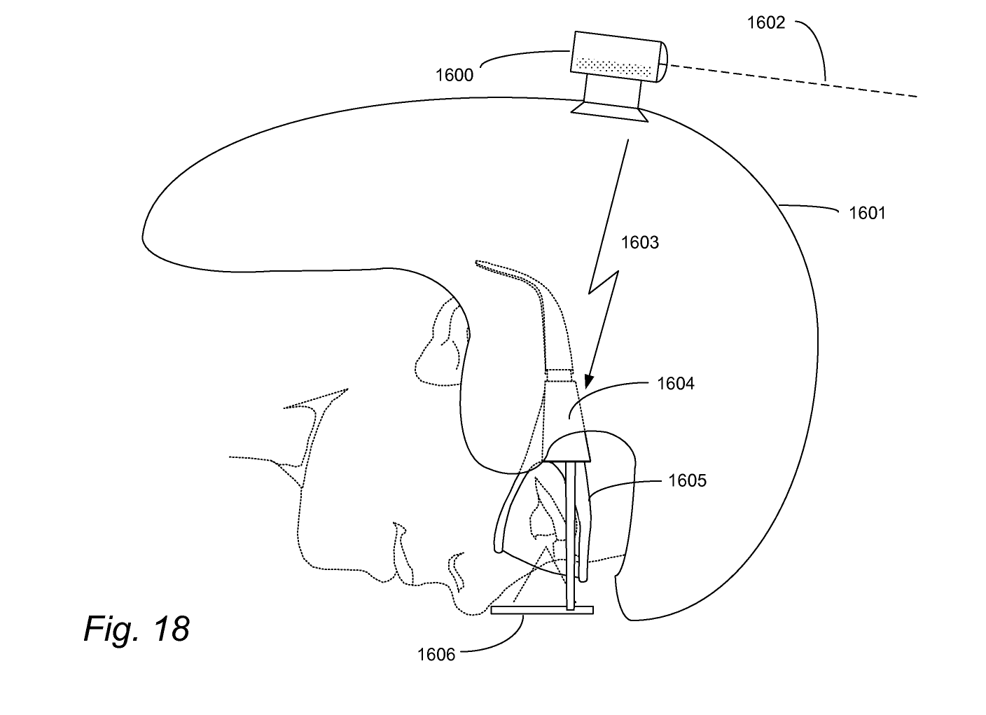

FIG. 18 shows an embodiment of the present invention comprising a miniature battery-operated video camera 1600 mounted to a racing helmet 1601 to provide a forward field of view 1602 when the racer's head is lowered into an aerodynamically efficient position. The video camera is constructed using any of the available low power imager chips that are used in portable cellular “smart” telephone units, for example. In a non-limiting example, the image signal from the camera 1603 is wirelessly communicated using additional wireless communications link hardware and software enclosed in the camera housing to a battery-operated receiver/processor 1604 mounted on special racing eyewear 1605. The received image is displayed on a miniature video display device 1606 that presents the forward scene directly to the bicyclist's eye. The miniature video display device is a low-power unit such as is typically used as a viewfinder in a modern electronic camera system.

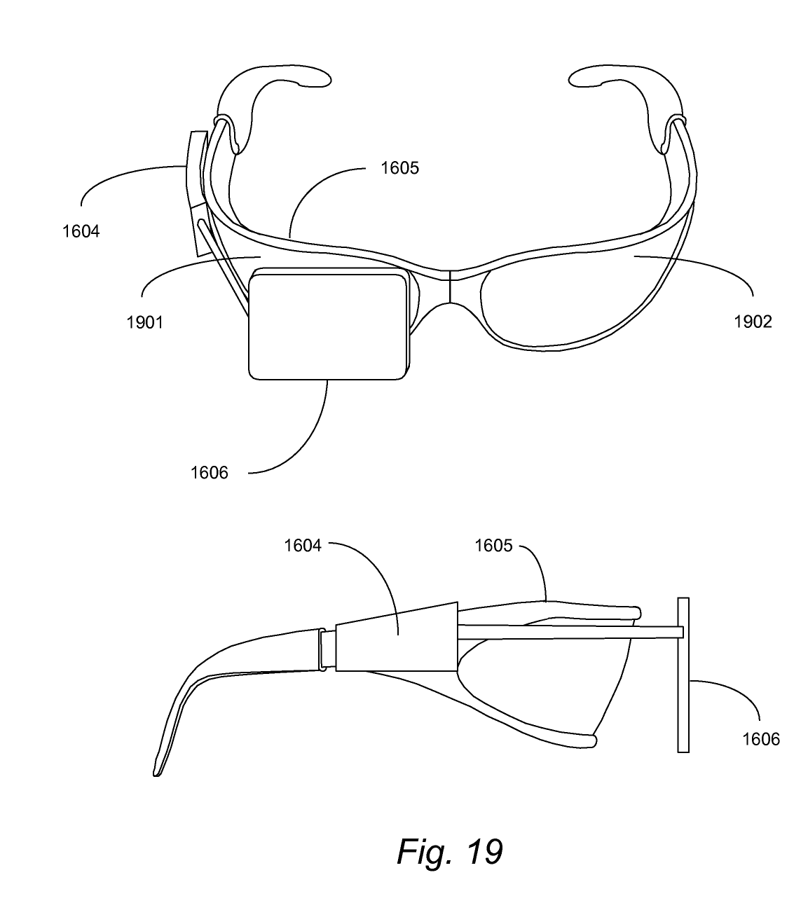

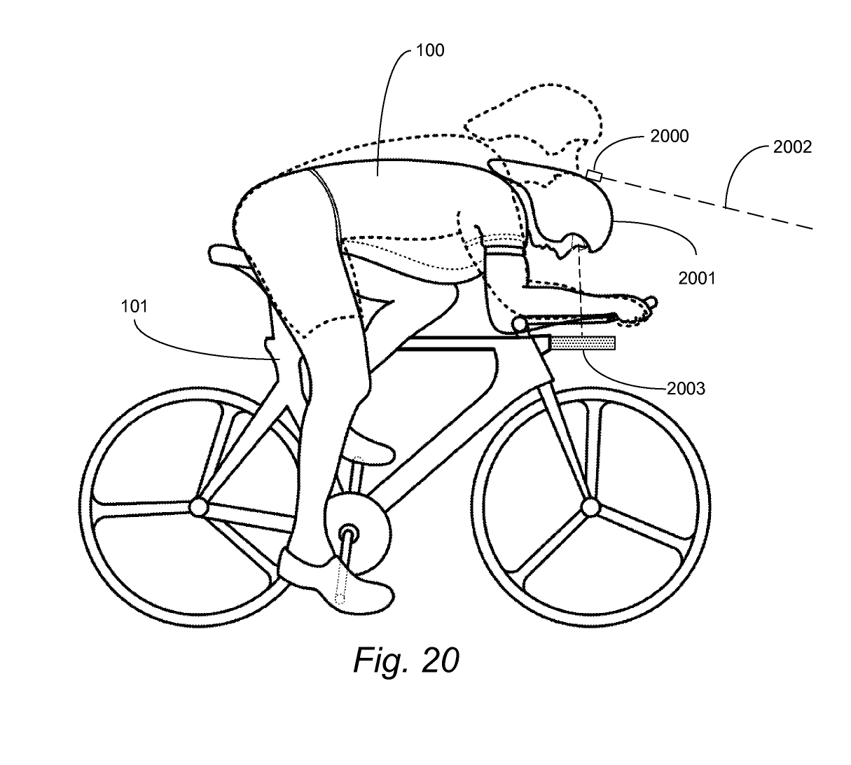

FIG. 19 shows frontal and right side views of the special racing eyewear depicted in FIG. 16. The eyewear includes right 1901 and left 1902 lenses, a battery-operated receiver/processor 1604 and a miniature video display screen 1606 mounted in front of one of the lenses.

FIG. 20 shows an alternative embodiment of the present invention including a rider 100 positioned on a bicycle 101 in a more aerodynamically efficient riding position wherein the rider's head and torso are lowered to reduce the effective frontal area and achieve a configuration having a smaller coefficient of drag, wherein the miniature video camera 2000 attached to the bicyclist's racing helmet 2001 covers a forward field of view 2002. In this embodiment, the image signal from the video camera is communicated to a battery-operated video display device 2003 which is mounted to the frame of the bicycle 101 in such a position as to provide direct viewing of the forward scene by the rider 100. The frame-mounted video display device can be constructed using a back-illuminated liquid-crystal display (LCD) screen as is used in portable computers, tablet computers and “smart” cellular telephone units. In another embodiment of the invention (not shown) the video camera is also affixed to the frame of the bicycle.

What is claimed is:

1. A bicycle racing shoe comprising:
a. a flat sole,
b. a pedal coupling member mounted directly to said flat sole,
c. a molding compound formed over at least the front half of said flat sole to create an aerodynamically shaped surface enclosing the pedal coupling member.

2. The bicycle racing shoe of claim 1 wherein the molding compound is selected from epoxies, crepe sole shoe materials, urethane compounds and room temperature vulcanizing silicone compounds.

3. A body fairing comprising:
a. at least one inflatable section having a top and a bottom and worn against a bicyclist's frontal torso to provide an enhanced aerodynamic shape to the bicyclist's torso,
b. wherein the inflatable section is inflated using an integrated coupling and valve assembly,
c. wherein the inflatable section is attached to said bicyclist's torso using shoulder straps attached to the top of said inflatable section that attach to a waist strap attached to the bottom of said inflatable section,
d. wherein the inflatable section or sections each have a quick fill cap and anti-splash opening for inputting water or electrolyte/food material.

4. The body fairing of claim 3 further comprising at least one inflatable section worn against a bicyclist's rear torso.

5. A bicycle racing goggle comprising:
a. at least one transparent lens mounted in a frame worn on a bicyclist's head,
b. an optical device attached to said lens that provides forward vision to the bicyclist when the bicyclist's head faces downward in order to allow the bicyclist to maintain an aerodynamically efficient riding position.

6. The bicycle racing goggle of claim 5 wherein the optical device is a prism.

7. The bicycle racing goggle of claim 5 wherein the optical device is a Fresnel lens.

8. The bicycle racing goggle of claim 5 wherein the optical device is a wide-angle lens.

9. A vision system for a bicyclist comprising:
a. a video camera operably mounted on a rear surface of a racing helmet worn on a bicyclist's head so as to provide an image of the field of view ahead of the bicycle when the bicyclist's head is lowered in order to allow the bicyclist to maintain an aerodynamically efficient riding position,
b. a video display device that communicates with said video camera and provides the image from said video camera to the bicyclist when the bicyclist's head is lowered.

10. The vision system of claim 9 wherein the video display device is attached to the racing helmet worn by the bicyclist.

11. The vision system of claim 9 wherein the video display device is attached to eyewear worn by the bicyclist.

12. The vision system of claim 9 wherein the video display device is mounted to the frame of a bicycle ridden by the bicyclist.

13. The vision system of claim 9 wherein the video display device communicates with the video camera using electronic cables.

14. The vision system of claim 9 wherein the video display device communicates with the video camera using a wireless communications link.
